(19) United States
(12) Patent Application Publication
(10) Pub. No.: US 2007/0226814 A1
(43) Pub. Date: Sep. 27, 2007

(54) Fish Food for Aquatic Farms, Based on Fermented Polyunsaturated Fatty Acids

(76) Inventors: Thomas Kiy, Frankfurt/Main (DE); Stephan Hausmanns, Wiesbaden (DE); Jurgen Haffner, Ingelheim (DE)

Correspondence Address: HAMMER & HANF, PC, 3125 SPRINGBANK LANE, SUITE G, CHARLOTTE, NC, 28226, US

(21) Appl. No.: 11/568,465

(22) PCT Filed: May 2, 2005
(86) PCT No.: PCT/EP05/04745

§ 371 Date: Jun. 11, 2007

(30) Foreign Application Priority Data
May 3, 2004 (DE) 10 2004 022 015.8

(51) Int. Cl. A01K 67/00; A23D 7/00

(52) U.S. Cl. 800/8; 426/608

(57) ABSTRACT

This invention relates to a feed containing at least one biomass that has been obtained by fermentation or oil extracts obtained from the biomass. The feed is rich in long-chain, polyunsaturated omega-3 fatty acids and can contribute to the maintenance of the natural fatty acid profile in farmed fish or can restore the profile.

The present invention relates to feed for aquatic farms on the basis of polyunsaturated fatty acids obtained by fermentation and in particular suitable for use as feed for fish and crustaceans, preferably for salmon.

The rearing of edible fish and in particular of salmon in open or closed aquatic farms (fish farming in a controlled environment) has long been state of the art. Approximately half of the salmon, 40 percent of the molluscs and 65 percent of the freshwater fish consumed today is produced in such farms at present.

Especially in the meat of salmon, but also of other seawater fish cultured in aquatic farms, such as hake, adult cod and young cod, as well as in crustaceans, e.g., shrimps, there is a high proportion of long-chain polyunsaturated omega-3 fatty acids, in particular C22:n3 docosahexaenoic acid (DHA), which are very valuable and of vital importance for the health of the animals and also for human nutrition. Thus, for example, the feeding of long-chain polyunsaturated omega-3 fatty acids for the therapy of the “Hitra” disease in fish is known from the EP patent publication 0 322 114. The PCT publication WO 00/62625 describes the superior bioavailability of the fish pigment astaxanthin with simultaneous feeding of long-chain polyunsaturated omega-3 fatty acids.

The natural producers of these long-chain polyunsaturated omega-3 fatty acids are marine microalgae, whose fat concentrates through the food chain in free-living fish. In aquatic farms, this natural source of omega-3 fatty acids is non-existent, for which reason it is necessary to resort to fish food enriched with fish oil or fish meal. However, the resources of these omega-3 rich fish food additives, which are obtained predominantly from fish waste, are increasingly limited as a result of the overfishing of the seas and the increasingly better utilization of the by-catch, in particular against the background of the strongly growing amount of farmed fish produced from aquacultures.

In order to solve this problem, for example the EP patent publication 1 346 647 A1 and the WO publication 03/075677 A1 describe the feeding of vegetable oils or extracts, which are rich in medium-chain omega-3 fatty acids, such as, e.g., α-linolenic acid. Among others, the following are cited as sources: soybean meal, wheat, sunflower meal and specific vegetable oils such as linseed oil, rapeseed oil or soy oil.

However, the previously described prior art is disadvantageous in that these sources of fatty acids are not converted or only converted to a very limited extent into the long-chain omega-3 fatty acids, such as, e.g., DHA. As a consequence, there is an undersupply of farmed fish with long-chain polyunsaturated omega-3 fatty acids, which, in turn, leads to an unnatural change of the fatty acid spectrum in fish, which is not accepted by the consumer.

In view of the prior art, it is therefore the object of the present invention to provide a fish food, which does not lead to a change in the fatty acid spectrum in farmed fish and through which a reestablishment and preservation of the natural proportion of long-chain polyunsaturated omega-3 fatty acids in fish can be achieved.

This and further not explicitly described tasks, which can, however, be derived or deduced without difficulty from the relations discussed in the introduction, are achieved by the object defined in the claims of the present invention.

An advantageous feed is provided by the preparation defined in claim 1. The feed according to the invention includes at least one biomass that has been obtained by fermentation or oil extracts obtained therefrom, which is rich in long-chain polyunsaturated n-3 fatty acid. Furthermore, the feed may contain the customary additives, such as e.g. vegetable meal, vitamins, mineral salts, antioxidants and further feed additives known to the person skilled in the art.

By making available a largely natural source of omega-3 fatty acids on the basis of reusable raw materials as fish food, above object is solved in a surprisingly simple manner. Moreover, ethical objections and objections based on environmental protection concerns against the use of fish meal as feed for fish are also dispelled in this way.

In particular, the present invention therefore relates to a feed for fish, which is based on a biomass comprised of microorganisms, in which the biomass contains at least 20 area % of DHA, relative to the total fatty acid content (TFA, Total Fatty Acids). In particular, the biomass per se can be the feed without further processing.

According to another preferred aspect, the present invention also concerns a feed, which is produced by mixing conventional feeds with the DHA-containing biomass, where, due to the dilution effect, higher DHA contents of the biomass are preferred.

It is further also preferred when the biomass also contains EPA in addition to DHA.

Moreover, a lipid extract from the biomass can also be used as feed instead of the biomass, the lipid extract being preferably mixed with conventional feeds.

In the sense of the present invention, omega-3 fatty acids (n-3 fatty acids) are taken to mean long-chain polyunsaturated fatty acids (PUFAs) having a chain length>12 carbon atoms comprising at least two or more double bonds, in which the first of the at least two or more double bonds is constituted between the carbon atoms C3 and C4, starting from the alkyl end. According to the invention, the n-3 fatty acids can be present as free fatty acids, esters, triglycerides, phospholipids or as other derivatives or be converted and concentrated by means of chemical or biocatalytic transesterification, for example with the help of suitable enzymes (lipases). All of these substances are summarized hereinafter by the terms n-3 fatty acid or n-3 active compounds, with the terms being used synonymously.

Examples of known medium-chain or long-chain n-3 fatty acids are found in the following table:

| | | Trivial name, |
|---|---|---|
| | IUPAC name | abbreviation |
| C18:3 | all-cis-9,12,15-octadecatrienoic acid | α-linolenic |
| | | acid, ALA |
| C18:4 | all-cis-6,9,12,15-octadecatetraenoic acid | stearidonic |
| | | acid |
| C20:3 | all-cis-11,14,17-eicosatrienoic acid | |
| C20:4 | all-cis-8,11,14,17-eicosatetraenoic acid | ETA |
| C20:5 | all-cis-5,8,11,14,17-eicosapentaenoic acid | timnodonic |
| | | acid, EPA |
| C22:3 | all-cis-13,16,19-docosatrienoic acid | |
| C22:5 | all-cis-7,10,13,16,19-docosapentaenoic acid | DPA |
| C22:6 | all-cis-4,7,10,13,16,19-docosahexaenoic acid | DHA |

Preference within the scope of this invention is given to biomasses obtained by fermentation or oil extracts obtained therefrom, which contain docosahexaenoic acid and eicosapentaenoic acid, with DHA being particularly preferred.

Preferred Microorganisms used as sources for DHA are organisms belonging to the Stramenopiles (or Labyrinthulomycota), particularly preferably to the order Thraustochytriales, (Thraustchytriidea), in particular to the genera Schizochytrium, Thraustochytrium and Ulkenia, and also Dinoflagellates (Dinophyta), preferably Crypthecodinium, in particular C. cohnii., which are preferably suitable for producing DHA at a concentration of at least 20 area-% of TFA (Total Fatty Acids), preferably at least 30 area-% of TFA, and particularly preferably at least 40 area-% of TFA DHA. In this case, with respect to the production of n-3 fatty acids, the following patent publications are incorporated in particular by reference: WO-A-91/07498, WO-A-91/11918, WO-A-96/33263 and WO-A-98/03671.

Further suitable sources of EPA and/or DHA are also, e.g. microalgae such as Euglena (JP-A-60-196157), Nannochloropsis, Phaeodactylum and others (Tonon et al., Long chain polyunsaturated fatty acid production and partitioning to triacylglycerols in four microalgae.

Phytochemistry 2002, 15-24), but also bacteria, preferably e.g. Shewanella, Vibrio or Moritella (Cho and Mo, Screening and characterization of eicosapentaenoic acid-producing marine bacteria, Biotechnology Letters 1999, 215-218; JP-A-2000/245442; JP-A-63-216490, JP-A-2001/309797).

A further possible source of n-3 fatty acids are transgenic organisms, preferably microorganisms and plants.

Particular preference is given to biomasses or lipid extracts from Ulkenia sp.

It was particularly surprising that a composition of such low complexity, i.e. based on only one type of microorganism, is sufficient to preserve the natural fatty acid spectrum in fish, even though free-living animals feed on an extraordinarily complex mixture of a great variety of prey species.

The biomass should therefore be comprised of at least 10% by weight, preferably at least 50% by weight and particularly preferably at least 90% by weight, very particularly preferably at least 99% by weight and most preferably exclusively of Ulkenia sp.

In this respect, it was surprisingly found that the by far best source for the biomass according to the invention is Ulkenia, which surpasses all the other microorganisms investigated.

This applies not only to the growth but also to the vitality and the largely natural fatty acid spectrum of the fish fed according to the invention.

The omega-3 rich biomass can be used directly as feed in aquacultures or admixed in conventional feeds. The flowability of the biomass can thereby be adapted by means of different methods (comminution, desiccation ratio, granulation) known to the person skilled in the art. The biomass can also be fed in a moist or semi-moist condition.

In the case of lipid extracts, mixing with common fish feeds is preferred.

Fish feeds are understood to be feeds which are usually employed in aquaculture, thus being suitable for fish as well as for, e.g., crustaceans.

The invention also concerns animals, especially fish and/or crustaceans, which are produced with the feed according to the invention.

1. Feed comprising at least one biomass consisting of microorganisms, wherein the biomass contains at least 20 area % of DHA, relative to the total fatty acid content (TFA, Total Fatty Acids), and, where applicable, customary fish feed additives.

2. Feed according to claim 1, which additionally contains EPA.

3. Feed according to claim 1, characterized in that the biomass is comprised of at least 10% by weight of Ulkenia sp.

4. Feed according to claim 1, characterized in that, instead of the biomass, a lipid extract of the biomass is used.

5. Method for producing a farmed animal,
wherein said farmed animal is selected from the group consisting of: fish, crustaceans or combinations thereof;
where said farmed animal has a natural fatty acid spectrum;
where the natural fatty acid spectrum of the animals is preserved, comprising the steps of:
providing a feed according to claim 1; and
feeding said feed to said farmed animal.

6. Fish and crustaceans obtained by a method according to claim 5.

7. Feed according to claim 2, characterized in that the biomass is comprised of at least 10% by weight of Ulkenia sp.

8. Feed according to claim 2, characterized in that, instead of the biomass, a lipid extract of the biomass is used.

9. Feed according to claim 3, characterized in that, instead of the biomass, a lipid extract of the biomass is used.

10. Feed according to claim 1, characterized in that the biomass is comprised of at least 50% by weight of Ulkenia sp.

11. Feed according to claim 1, characterized in that the biomass is comprised of at least 90% by weight of Ulkenia sp.

12. Feed according to claim 1, characterized in that the biomass is comprised of at least 99% by weight of Ulkenia sp.

13. Feed according to claim 1, characterized in that the biomass is comprised exclusively of Ulkenia sp.

14. Feed according to claim 2, characterized in that the biomass is comprised of at least 50% by weight of Ulkenia sp.

15. Feed according to claim 2, characterized in that the biomass is comprised of at least 90% by weight of Ulkenia sp.

16. Feed according to claim 2, characterized in that the biomass is comprised of at least 99% by weight of Ulkenia sp.

17. Feed according to claim 2, characterized in that the biomass is comprised exclusively of Ulkenia sp.

18. Method for producing a farmed animal,
wherein said farmed animal is selected from the group consisting of: fish, crustaceans or combinations thereof;
where said fanned animal has a natural fatty acid spectrum;
where the natural fatty acid spectrum of the animals is preserved, comprising the steps of:
providing a feed according to claim 2; and
feeding said feed to said farmed animal.

19. Method for producing a farmed animal,
wherein said fanned animal is selected from the group consisting of: fish, crustaceans or combinations thereof;,
where said farmed animal has a natural fatty acid spectrum;
where the natural fatty acid spectrum of the animals is preserved, comprising the steps of:
providing a feed according to claim 3; and
feeding said feed to said farmed animal.

20. Method for producing a farmed animal,
wherein said farmed animal is selected from the group consisting of: fish, crustaceans or combinations thereof;
where said farmed animal has a natural fatty acid spectrum;
where the natural fatty acid spectrum of the animals is preserved, comprising the steps of:
providing a feed according to claim 4; and
feeding said feed to said farmed animal.
